(19) United States
(12) Patent Application Publication
(10) Pub. No.: US 2022/0133669 A1
(43) Pub. Date: May 5, 2022

(54) PHARMACEUTICAL COMPOSITIONS COMPRISING 15-HETRE AND METHODS OF USE THEREOF

(71) Applicant: DS Biopharma Limited, Dublin (IE)

(72) Inventors: John Climax, Dublin (IE); David Coughlan, Dublin (IE); Bill Downes, Dublin (IE); Tien Nghiem, Dublin (IE); Markus Weissbach, Dublin (IE); Moayed Hamza, Dublin (IE)

(21) Appl. No.: 17/514,381

(22) Filed: Oct. 29, 2021

Related U.S. Application Data
(60) Provisional application No. 63/107,549, filed on Oct. 30, 2020.
(60) Provisional application No. 63/107,563, filed on Oct. 30, 2020.

(51) Int. Cl. A61K 31/202; A61K 9/00; A61K 45/06; A61P 31/14; A61P 3/10

(52) U.S. Cl. CPC A61K 31/202; A61K 9/0053; A61P 3/10; A61P 31/14; A61K 45/06

(57) ABSTRACT

Provided herein are pharmaceutical compositions comprising 15-HETrE and methods of using the same to treat a variety of conditions and disorders.

### PRIORITY CLAIM

This application claims priority to U.S. Provisional Patent Application Ser. No. 63/107,549 filed Oct. 30, 2020, and U.S. Provisional Patent Application Ser. No. 63/107,563 filed Oct. 30, 2020, the entire contents of which are incorporated herein by reference.

### TECHNICAL FIELD

The present disclosure generally relates generally to pharmaceutical compositions comprising 15-hydroxyeicosatrienoic acid (15-HETrE) and methods of using the same.

### BACKGROUND

15-hydroxyeicosatrienoic acid (15-HETrE) is an in vivo metabolite resulting from 15-lipoxygenation of dihomo gamma linolenic acid (DGLA). DGLA is an essential fatty acid found naturally in the body as the elongation product of gamma linolenic acid (GLA). GLA is in turn a desaturation product of linoleic acid.

### SUMMARY

The present disclosure provides compositions comprising 15-HETrE, and methods of using the same to treat and/or prevent a disease and/or condition in a subject in need thereof.

In some aspects, the present disclosure provides a composition comprising 15-HETrE and or more active agents selected from the group consisting of a skin agent, a renal agent, a liver agent, a lung agent, a heart agent, a pancreas agent or anti-diabetic agent, a blood agent, a colon agent, and an anti-viral agent.

In certain embodiments, the composition is formulated for intravenous administration, oral administration, or intranasal administration.

In another aspect, the present disclosure provides an orally deliverable composition comprising 15-HETrE.

In some embodiments, the orally deliverable composition comprises one or more therapeutic agents selected from the group consisting of a skin agent, a renal agent, a liver agent, a lung agent, a heart agent, a pancreas agent or anti-diabetic agent, a blood agent, a colon agent, and an anti-viral agent.

In yet another aspect, the present disclosure provides a method of treating or preventing a disease in a subject in need thereof, the method comprising administering to the subject a composition comprising 15-HETrE, wherein the disease is selected from the group consisting of a skin disease, kidney disease, liver disease, spleen disease, lung disease, heart disease, pancreas disease, blood disease, colon disease, and a viral disease.

In some embodiments, the 15-HETrE is in free acid form and/or a pharmaceutically acceptable ester, derivative, conjugate, or salt thereof, or mixtures of any of the foregoing. In certain embodiments, the 15-HETrE is 15-HETrE ethyl ester.

In some embodiments, the 15-HETrE is present in the composition in an amount up to about 1500 mg. In some embodiments, the composition comprises up to about 4 g of 15-HETrE. In yet another embodiment, the composition comprises up to about 2 g of 15-HETrE. In another embodiment, the composition comprises about 2 g to about 4 g of 15-HETrE.

In some embodiments, the 15-HETrE represents at least about 80%, by weight, of all fatty acids present in the composition.

In some embodiments, the composition is present in a solid dosage form. In yet another embodiment, the solid dosage form comprises a capsule. In certain embodiments, the composition is present in a liquid dosage form or semi-solid dosage form. In some embodiments, the liquid dosage form or semi-solid dosage form comprises a solution or an emulsion.

In some embodiments, the subject is administered one or more therapeutic agents selected from the group consisting of a skin agent, a renal agent, a liver agent, a lung agent, a heart agent, a pancreas agent or anti-diabetic agent, a blood agent, a colon agent, and anti-viral agent.

In some embodiments, the composition is administered to the subject intravenously, orally, or intranasally.

In certain embodiments, the skin disease is selected from the group consisting of acne, atopic dermatitis, bacterial infections, dermatitis, dry skin, eczema, fungal infections, photoprotection, psoriasis, pruritus/itch, photoprotection, radiation protection, seborrheic dermatitis, shingles, vasculitis, viral infections, and wrinkles.

In another embodiment, the renal disease is selected from the group consisting of polycystic kidney disease, chronic kidney disease, acute kidney failure, kidney infection (pyelonephritis), and kidney stones.

In certain embodiments, the liver disease is selected from the group consisting of steatohepatitis, alcoholic hepatitis, liver toxicity, viral infection of the liver, viral hepatitis, autoimmune hepatitis, cryptogenic cirrhosis, hepatic necrosis following hypoperfusion, and hepatitis resulting from other disease, and secondary NASH.

In some embodiments, the spleen disease is selected from the group consisting of splenomegaly, spleen cancer, asplenia, spleen trauma, idiopathic purpura, Felty's syndrome, Hodgkin's disease, and immune-mediated destruction of the spleen.

In yet another embodiment, the lung disease is selected from the group consisting of reactive airway disease, asthma, emphysema, COPD, respiratory tract infection, pleural cavity disease, pulmonary vascular disease, pneumonia, pulmonary embolism, lung cancer, and silicosis.

In some embodiments, the heart disease is selected from the group consisting of arrhythmia, atherosclerosis, cardiomyopathy, congenital heart defects, coronary artery disease (CAD), myocardial infarction, high blood pressure, cardia arrest, congestive heart failure, peripheral artery diseases, stroke, and heart infections.

In certain embodiments, the pancreas disease is selected from the group consisting of type 1 diabetes, type 2 diabetes, pancreatitis, and pancreatic cancer.

In some embodiments, the blood disease is selected from the group consisting of anemia, hemoglobinopathy, sickle cell disease, alpha-thalassemia, beta-thalassemia, Hodgkin's lymphoma, non-Hodgkin's lymphoma, leukemia, and multiple myeloma.

In another embodiment, the colon disease is selected from the group consisting of inflammation, ulcerative colitis, colon cancer, colonic polyps, Crohn's disease, diverticulosis, diverticulitis, intestinal obstructions, and irritable bowel syndrome.

In some embodiments, the viral disease is caused by a coronavirus. In certain of these embodiments, the coronavirus is selected from the group consisting of SARS-COV, MERS-COV, and SARS-COV-2.

### BRIEF DESCRIPTION OF THE DRAWINGS

Many aspects of the present disclosure can be better understood with reference to the following drawings.

Figures (FIGS. 1A-1D present data from an in vivo study examining tissue distribution of 15-HETrE (ng/g) in heart, kidney, liver, and spleen following oral administration of 15-HETrE, in accordance with embodiments of the present disclosure.

FIGS. 2A-2D present data from an in vivo study examining tissue distribution of 15-HETrE (ng/g) in lung, plasma, skin, and colon following oral administration of 15-HETrE, in accordance with embodiments of the present disclosure.

FIG. 3 is a schematic of the cytotoxicity pretest study of Example 6 in accordance with embodiments of the present disclosure.

FIGS. 4A-4B present data from the cytotoxicity pretest study of Example 6 examining cytotoxic threshold of 15(S)-HETrE in islet microtissues in accordance with embodiments of the present disclosure.

FIG. 5 is a schematic of the 15-HETrE efficacy study of Example 6 in accordance with embodiments of the present disclosure.

FIGS. 6A-6H present data from the 15-HETrE study of Example 6 examining the effects of 15(S)-HETrE on human islet microtissue function under conditions of glucotoxicity and cytokine stress in accordance with embodiments of the present disclosure.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the present disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

### Compositions

A. 15-HETrE

In some aspects, the present disclosure provides pharmaceutical compositions comprising 15-HETrE.

In some embodiments, compositions of the invention comprise 15-HETrE as an active ingredient. 15-HETrE is the abbreviation for 15-hydroxy-eicosa-8(Z),11(Z),13(E)-trienoic acid, a metabolite of DGLA (dihomo-gamma linolenic acid). As used herein, the term “15-HETrE” refers to 15-HETrE in its free acid form (e.g., 15-hydroxy-eicosa-8(Z),11(Z),13(E)-trienoic acid), a pharmaceutically acceptable ester, derivative, conjugate, or salt thereof, or mixtures of any of the foregoing. The term “pharmaceutically acceptable” in the present context means that the substance in question does not produce unacceptable toxicity to the subject or interaction with other components of the composition.

In some embodiments, the 15-HERE comprises a C₁-C₅ alkyl ester of 15-HERE. In one embodiment, the 15-HERE comprises 15-HETrE methyl ester, 15-HETrE propyl ester, or 15-HETrE butyl ester.

In some embodiments, the 15-HETrE comprises 15-hydroxy-eicosa-8(Z),11(Z),13(E)-trienoic acid ethyl ester. In this embodiment, the 15-HETrE is in the form of an ethyl ester (also referred to herein as 15-HETrE EE or 15-HETrE ethyl ester). As shown in the Examples, 15-HETrE ethyl ester can be synthesized from 15-HETrE free acid.

In another embodiment, the 15-HETrE comprises lithium 15-HETrE, mono, di- or triglyceride 15-HETrE.

In one embodiment, the compositions comprise a therapeutically effective amount of 15-HETrE. In one embodiment, the pharmaceutical composition comprises about 0.1% to about 99%, about 1% to about 95%, about 5% to about 90% by weight of 15-HETrE.

In one embodiment, the pharmaceutical composition comprises at least about 70?, at least about 80% or at least about 90%, by weight, of 15-HETrE. In one embodiment, the pharmaceutical composition comprises at least about 50%, at least about 60%, at least about 70%, at least about 80%, or at least about 90%, by weight of 15-HETrE.

In one embodiment, 15-HETrE is present in a composition of the present disclosure and comprises at least 90% by weight 15-HERE (as the term “15-HETrE” is defined and exemplified herein). 15-HETrE compositions can comprise even higher purity 15-HETrE, for example at least 95% by weight 15-HETrE, at least 96% by weight 15-HETrE, at least 97% by weight 15-HETrE, at least 98% by weight 15-HETrE, at least 99% by weight 15-HETrE, or 100% by weight 15-HETrE, wherein the 15-HERE is any form of 15-HETrE as set forth herein. The purity of 15-HETrE can further be defined (e.g., impurity profile) by any of the descriptions of 15-HETrE provided herein.

In various embodiments, 15-HETrE is present in a composition of the present disclosure in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg, 5600 mg, about 5700 mg, about 5800 mg, about 5900 mg, about 6000 mg, about 6100 mg, about 6200 mg, about 6300 mg, about 6400 mg, about 6500 mg, 6600 mg, about 6700 mg, about 6800 mg, about 6900 mg, about 7000 mg, about 7100 mg, about 7200 mg, about 7300 mg, about 7400 mg, about 7500 mg, 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, about 9000 mg, about 10000 mg, about 10100 mg, about 10200 mg, about 10300 mg, about 10400 mg, about 10500 mg, about 10600 mg, about 10700 mg, about 10800 mg, about 10900 mg, about 11000 mg, about 11100 mg, about 11200 mg, about 11300 mg, about 11400 mg, about 11500 mg, about 11600 mg, about 11700 mg, about 11800 mg, about 11900 mg, or about 12000 mg.

In one embodiment, a composition of the present disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, of fatty acids other than 15-HETrE.

In one embodiment, a composition of the present disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-6 fatty acids such as linoleic acid, gamma linolenic acid (GLA), dihomo gamma linolenic acid (DGLA) or derivatives thereof. In other embodiments there is substantially no, or no other omega-6 fatty acids present.

In another embodiment, 15-HETrE represents at least about 30%, about 40%, about 50%, at least about 60%, at least about 70%, at least about 80%, at least about 90%, at least about 95%, at least about 96%, at least about 97%, at least about 98%, at least about 99%, or 100%, by weight, of all fatty acids present in a composition of the present disclosure.

B. 15-HETrE Esters

In some aspects, a pharmaceutical composition of the present disclosure comprises 15-HETrE in the form of an alkyl ester, including, but not limited to, a C₁-C₅ alkyl ester of 15-HETrE. In some embodiments, the 15-HETrE is in the form of 15-HETrE methyl ester, 15-HETrE ethyl ester, 15-HETrE propyl ester, or 15-HETrE butyl ester.

In some embodiments, the pharmaceutical composition comprises 15-HETrE ethyl ester (15-hydroxy-eicosa-8(Z),11(Z),13(E)-trienoic acid ethyl ester, 15-HETrE EE, or 15-HETrE ethyl ester). 15-HETrE ethyl ester is a chiral molecule and may be used in the (S)- or (R)-enantiomeric form, or as a racemic mixture. As used herein, “15-HETrE ethyl ester” includes all such forms, with no limitation as to stereospecificity. In some embodiments, 15-HETrE ethyl ester comprises 15(S)-HETrE ethyl ester, 15(R)-HETrE ethyl ester, or a mixture thereof.

In some embodiments, a pharmaceutical composition of the present disclosure comprises a therapeutically effective amount of 15-HETrE ethyl ester. In one embodiment, the pharmaceutical composition comprises about 0.1% to about 99%, about 1% to about 95%, about 5% to about 90% by weight of 15-HETrE ethyl ester. In some embodiments, the pharmaceutical composition comprises at least about 50%, at least about 60%, at least about 70%, at least about 80%, or at least about 90%, by weight of 15-HETrE ethyl ester. In some embodiments, the pharmaceutical compositions can comprise an even higher purity of 15-HETrE ethyl ester, for example, at least 95% by weight, at least 96% by weight, at least 97% by weight, at least 98% by weight, at least 99% by weight, or 100% by weight 15-HETrE ethyl ester.

In some embodiments, 15-HETrE ethyl ester is present in a composition of the present disclosure in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg, 5600 mg, about 5700 mg, about 5800 mg, about 5900 mg, about 6000 mg, about 6100 mg, about 6200 mg, about 6300 mg, about 6400 mg, about 6500 mg, 6600 mg, about 6700 mg, about 6800 mg, about 6900 mg, about 7000 mg, about 7100 mg, about 7200 mg, about 7300 mg, about 7400 mg, about 7500 mg, 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, about 9000 mg, about 10000 mg, about 10100 mg, about 10200 mg, about 10300 mg, about 10400 mg, about 10500 mg, about 10600 mg, about 10700 mg, about 10800 mg, about 10900 mg, about 11000 mg, about 11100 mg, about 11200 mg, about 11300 mg, about 11400 mg, about 11500 mg, about 11600 mg, about 11700 mg, about 11800 mg, about 11900 mg, or about 12000 mg.

In some embodiments, the pharmaceutical composition contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, of fatty acids other than 15-HETrE ethyl ester.

In one embodiment, a composition of the present disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-6 fatty acids such as linoleic acid, gamma linolenic acid (GLA), dihomo gamma linolenic acid (DGLA) or derivatives thereof. In other embodiments there is substantially no, or no other omega-6 fatty acids present.

Alkyl esters (e.g., ethyl ester) according to the present disclosure may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002). For instance, and as shown in further details in the Examples, 15-HETrE ethyl ester can be prepared from 15-HETrE free acid with the addition of a suitable esterification agent.

C. Salts and Other Derivatives

Salts, hydrates, solvate, amides, enantiomers, isomers, tautomers, polymorphs, prodrugs, and derivatives of any of the foregoing drugs may be used in accordance with the present disclosure and may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable salts,” or “salts,” include the salt of a drug prepared from formic, acetic, propionic, succinic, glycolic; gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids.

In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In another embodiment, base addition salts are prepared from the free acid forms using conventional methodology involving reaction of the free acid with a suitable base.

In other embodiments, an acid addition salt is reconverted to the free base by treatment with a suitable base. In a further embodiment, the acid addition salts are halide salts, which are prepared using hydrochloric or hydrobromic acids. In still other embodiments, the basic salts are alkali metal salts, e.g., sodium salt. In other embodiments, a base addition salt is reconverted to the free acid by treatment with a suitable acid.

In some embodiments, the present disclosure provides a salt of 15-HETrE acid. In some embodiments, the salt is a sodium salt. Salts of 15-HETrE can be formed by any method known to one of skill in the art including as described in WO/2015/071766, which is incorporated by reference in its entirety.

D. Dosage Forms

In some aspects, pharmaceutical compositions comprising 15-HETrE and/or derivative thereof according to embodiments of the present disclosure can have various formulations for different routes of administration, including, but not limited to, oral formulations, injectable formulations, and liquid formulations.

In one embodiment, compositions of the present disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

In some embodiments, compositions of the present disclosure are injectable formulations or are formulated for injections through various administration routes, including, but not limited to, subcutaneous administration, intravenous administration, intraperitoneal administration, intramuscular administration, intradermal administration, and intrathecal administration. In some embodiments, the pharmaceutical compositions comprising 15-HETRE and/or derivative thereof is in a liquid formulation, for example, in the form of an emulsion, for intravenous administration.

In some embodiments, the pharmaceutical composition is formulated for intravenous injection. In yet another embodiment, the pharmaceutical composition is formulated for oral administration. In certain embodiments, the pharmaceutical composition is formulated for intranasal administration.

In some embodiments, compositions of the present disclosure are present in the form of solid dosage forms. Non-limiting examples of suitable solid dosage forms include tablets (e.g. suspension tablets, bite suspension tablets, rapid dispersion tablets, chewable tablets, melt tablets, effervescent tablets, bilayer tablets, etc), caplets, capsules (e.g. a soft or a hard gelatin or non-gelatin capsule filled with solid and/or liquids), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration.

In some embodiments, compositions of the present disclosure are present in the form of liquid or semi-solid dosage forms. Non-limiting examples of suitable liquid or semi-solid dosage forms include solutions and emulsions.

The compositions of the present disclosure can be formulated to have modified rates of release. Suitable modified-release formulations include those that exhibit a delayed- or extended-release. An “extended-release” formulation can extend the period over which the pharmaceutically active compound is released or targeted to the desired site. A “delayed-release” formulation can be designed to delay the release of the pharmaceutically active compound for a specified period. Mechanisms can be dependent or independent of local pH in the stomach and/or intestine, and can also rely on local enzymatic activity to achieve the desired effect, Examples of modified-release formulations are known in the art and are described; for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566 and The Handbook of Pharmaceutical Controlled Release Technology, D. L. Wise (ed.), Marcel Decker, Inc., New York (2000); and also in Treatise on Controlled Drug Delivery: Fundamentals, Optimization, and Applications, A. Kydonieus (ed.), Marcel Decker, Inc., New York, (1992), the relevant contents of each of which are hereby incorporated by reference for this purpose.

15-HETrE and/or any other additional agent(s) (e.g., a skin agent) can be co-formulated in the same dosage unit, or can be individually formulated in separate dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In discussing the amount of 15-HETrE in a composition of the present disclosure, this may be split over several dosage forms. There is a limit as to the size for oral administration. If a subject is to be administered 1 to 4 g 15-HETrE a day, this may be by up to 4 capsules, each providing 1 g 15-HETrE.

In one embodiment, a composition of the present disclosure comprises one or more additional therapeutic agents dispersed or suspended in 15-HETrE, wherein the dispersion or suspension is present in a capsule (for example gelatin or HPMC capsule, or other non-gelatin capsules), sachet, or other dosage form or carrier as described herein. In another embodiment, the dispersion or suspension is substantially uniform. In still another embodiment, where co-administration of two or more dosage units is desired, the 15-HETrE is present in a first dosage unit, for example a suspension in a capsule, and the second agent (e.g., skin agent) is present in second dosage unit, for example a tablet. Optionally, any desired additional oral agent for a skin condition can be present in a third composition.

In another embodiment, composition(s) of the present disclosure can be in the form of liquid dosage forms or dose units to be imbibed directly or they can be mixed with food or beverage prior to ingestion. Non-limiting examples of suitable liquid dosage forms include solutions, suspension, elixirs, syrups, liquid aerosol formulations, and the like.

In some embodiments, compositions of the present disclosure are encapsulated in a capsule shell.

E. Excipients

Compositions of the present disclosure optionally comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition.

Compositions of the present disclosure optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition.

Compositions of the present disclosure optionally comprise one or more pharmaceutically acceptable disintegrants as excipients. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums. Such disintegrants, if present, typically comprise in total about 0.2% to about 30%, about 0.2% to about 10%, or about 0.2% to about 5%, of the total weight of the composition.

Compositions of the present disclosure optionally comprise one or more antioxidants. Illustrative antioxidants include sodium ascorbate and vitamin E (tocopherol). One or more antioxidants, if present, are typically present in a composition of the present disclosure in an amount of about 0.001% to about 5%, about 0.005% to about 2.5%, or about 0.01% to about 1%, by weight.

Compositions of the present disclosure optionally comprise one or more pharmaceutically acceptable binding agents or adhesives as excipients. Such binding agents and adhesives can impart sufficient cohesion to a powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

Compositions of the present disclosure optionally comprise one or more pharmaceutically acceptable wetting agents as excipients. Non-limiting examples of surfactants that can be used as wetting agents in compositions of the present disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Compositions of the present disclosure optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is an anti-adherent or glidant used, for example, to reduce formulation sticking to equipment surfaces and also to reduce static in the blend. Talc, if present, constitutes about 0.1% to about 10%, about 0.25% to about 5%, or about 0.5% to about 2%, of the total weight of the composition. Glidants can be used to promote powder flow of a solid formulation. Suitable glidants include colloidal silicon dioxide, starch, talc, tribasic calcium phosphate, powdered cellulose and magnesium trisilicate.

Compositions of the present disclosure optionally comprise one or more flavoring agents, sweetening agents, and/or colorants. Flavoring agents useful in the present disclosure include, without limitation, acacia syrup, alitame, anise, apple, aspartame, banana, Bavarian cream, berry, blackcurrant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cyclamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents that can be used in the present disclosure include, for example, acesulfame potassium (acesulfame K), alitame, aspartame, cyclamate, cyclamate, dextrose, isomalt, MagnaSweet®, maltitol, mannitol, neohesperidin DC, neotame, Prosweet® Powder, saccharin, sorbitol, stevia, sucralose, sucrose, tagatose, thaumatin, xylitol, and the like.

Flavoring agents, sweetening agents, and/or colorants can be present in compositions of the present disclosure in any suitable amount, for example about 0.01% to about 10%, about 0.1% to about 8%, or about 1% to about 5%, by weight.

Compositions of the present disclosure optionally comprise a suspending agent. Non-limiting illustrative examples of suitable suspending agents include silicon dioxide, bentonite, hydrated aluminum silicate (e.g., kaolin) and mixtures thereof. One or more suspending agents are optionally present in compositions of the present disclosure in a total amount of about 0.01% to about 3.0%, about 0.1% to about 2.0%, or about 0.25% to about 1.0%, by weight

The foregoing excipients can have multiple roles as is known in the art. For example, starch can serve as filler as well as a disintegrant. The classification of excipients above is not to be construed as limiting in any manner. Excipients categorized in any manner may also operate under various different categories of excipients as will be readily appreciated by one of ordinary skill in the art.

### Additional Therapeutic Agents

In some aspects, the pharmaceutical compositions comprising 15-HETrE and/or derivative thereof according to various embodiments of the present disclosure comprise one or more additional therapeutic agents or are used for co-administration regimens with one or more additional therapeutic agents.

In some embodiments, the one or more additional therapeutic agents may be formulated into the same pharmaceutical composition comprising 15-HETrE and/or derivative thereof, for example, as a single dosage unit or as multiple dosage units for coordinated, combination, or concomitant administration, or into separate pharmaceutical compositions for combinational therapy.

In some embodiments, the one or more additional therapeutic agents may be formulated as separate pharmaceutical compositions, for example, as a single dosage unit or as multiple dosage units, for co-administration with the pharmaceutical composition comprising 15-HETrE and/or derivative thereof.

In some embodiments, the one or more additional therapeutic agents comprises a skin agent, a renal agent, a liver agent, a lung agent, a heart agent, a pancreas or anti-diabetic agent, a blood agent, a colon agent, and/or an anti-viral agent.

In one embodiment, 15-HETrE and the one or more additional therapeutic agents are present in a composition of the present disclosure, or are co-administered in a weight ratio of 15-HETrE:additional agent of about 1:1000 to about 1000:1, about 1:500 to about 500:1, about 1:100 to about 100:1, about 1:50 to about 50:1, about 1:25 to about 25:1, about 1:10 to about 10:1, about 1:5 to about 5:1, about 1:4 to about 4:1 about 1:3 to about 3:1, about 1:2 to about 2:1 or about 1:1.

In some embodiments, the one or more additional therapeutic agents are formulated for administration to a subject in a range of from about 1 mg/kg to about 500 mg/kg, 10 mg/kg to about 150 mg/kg, from 30 mg/kg to about 120 mg/kg, from 60 mg/kg to about 90 mg/kg. In some embodiments, the one of more additional therapeutic agents are administered to the subject at a dose of about 15 mg/kg, about 30 mg/kg, about 45 mg/kg, about 60 mg/kg, about 75 mg/kg, about 90 mg/kg, about 105 mg/kg, about 120 mg/kg, about 135 mg/kg, or about 150 mg/kg. In some embodiments, the one of more additional therapeutic agents may be formulated for administration once or multiple times a day.

A. Skin Agents

In some embodiments, the one or more additional therapeutic agents comprises a skin agent. The interchangeable terms “skin agent” or “skin drug” (e.g., a second skin agent), herein refer to a drug or agent that is capable of treating, preventing, or reducing the risk of developing a skin disease or disorder, or a risk factor or symptom thereof, in a subject. In some embodiments, the second skin agent is an oral skin agent. In other embodiments, the second skin agent is a topical skin agent. In yet other embodiments, the second skin agent is an injectable skin agent. Oral skin agents herein can include, without limitation, antibiotics (e.g., dicloxacillin, erythromycin, and tetracycline), antifungal agents (e.g., fluconazole and itraconazole), antiviral agents (acyclovir (Zovirax), famciclovir (Famvir), and valacyclovir (Valtrex), corticosteroids (e.g., prednisone), immunosuppressants (e.g., azathioprine and methotrexate), biologics (e.g., adalimumab, etanercept, etanercept-szzs, inflimirnab, ixekizumab, secukinumab, and ustekinumab), and enzyme inhibitors (e.g., apremilast), resinoids (e.g., acitretin). Topical skin agents herein can include, without limitation antibacterials (e.g., mupirocin and clindamycin), anthralin, antifungal agents (e.g., clotrimazole, ketoconazole, and terbinafine), benzoyl peroxide, coal tar, corticosteroids, and retinoids (e.g., retin-A and Tazorac), and salicylic acid.

B. Renal Agents

In some embodiments, the one or more additional therapeutic agents comprises a renal agent. The interchangeable terms “renal agent” or “renal drug” (e.g., a second renal agent) herein refer to a drug or agent that is capable of treating, preventing, or reducing the risk of developing a kidney disease or disorder, or a risk factor or symptom thereof, in a subject. In some embodiments, the second renal agent is an oral renal agent. In other embodiments, the second renal agent is a topical renal agent. In yet other embodiments, the second renal agent is an injectable renal agent. Renal agents herein can include, without limitation, high blood pressure medications (e.g., angiotensin-converting enzyme (ACE) inhibitors, angiotensin II receptor blockers (ARBS), beta-blockers, calcium channel blockers, direct renin inhibitors, diuretics, and vasodilators), medications to lower cholesterol levels, medications to treat anemia (e.g., erythropoietin (rhEPO) and iron replacement therapy), medications to relieve swelling, and medications to protect bones (e.g., calcitriol, paricalcitol, doxercalciferol).

C. Liver Agents

In some embodiments, the one or more additional therapeutic agents comprises a liver agent. The interchangeable terms “liver agent” or “liver drug” (e.g., a second liver agent) herein refer to a drug or agent that is capable of treating, preventing, or reducing the risk of developing a liver disease or disorder, or a risk factor or symptom thereof, in a subject. In some embodiments, the second liver agent is an oral liver agent. In other embodiments, the second liver agent is a topical liver agent. In yet other embodiments, the second liver agent is an injectable liver agent. Liver agents herein can include, without limitation, L-Ornithine L-Aspartate, 5-HT3 receptor antagonist (e.g., ondansetron), herbal regimens (e.g., silymarin and glycyrrhiza), hepatitis medications, and albumin.

D. Lung Agents

In some embodiments, the one or more additional therapeutic agents comprises a lung agent. The interchangeable terms “lung agent” or “lung drug” (e.g., a second lung agent) herein refer to a drug or agent that is capable of treating, preventing, or reducing the risk of developing a lung disease or disorder, or a risk factor or symptom thereof, in a subject. In some embodiments, the second lung agent is an oral lung agent. In other embodiments, the second lung agent is a topical lung agent. In yet other embodiments, the second renal agent is an injectable lung agent. In still other embodiments, the second lung agent is an inhalable lung agent. Lung agents herein can include, without limitation, steroids (momestone, ciclesonide, fluticasone, budesonide, beclomethasone HFA, prednisone, methyl-prednisolone) leukotriene modifiers (e.g., zafirlukast, montelukast, and zileuton), theophylline, combination medications (e.g., fluticasone/salmeterol (Advair®), mometasone/formoterol (Dulera®), budesonide/formoterol (Symbicort®), bronchodilators (e.g., anticholinergics, beta-agonists, and combinations thereof), phosphodiesterase-4 inhibitors, corticosteroids, methylxanthines, and herbal supplements.

E. Heart Agents

In some embodiments, the one or more additional therapeutic agents comprises a heart agent. The interchangeable terms “heart agent” or “heart drug” (e.g., a second heart agent) herein refer to a drug or agent that is capable of treating, preventing, or reducing the risk of developing a heart disease or disorder, or a risk factor or symptom thereof, in a subject. In some embodiments, the second heart agent is an oral heart agent. In other embodiments, the second heart agent is a topical heart agent. In yet other embodiments, the second heart agent is an injectable heart agent. Heart agents herein can include, without limitation, high blood pressure medications (e.g., angiotensin-converting enzyme (ACE) inhibitors, angiotensin II receptor blockers (ARBS), beta-blockers, calcium channel blockers, direct renin inhibitors, diuretics, and vasodilators), medications to lower cholesterol levels, medications to treat heart failure (e.g., angiotensin-converting enzyme (ACE) inhibitors, angiotensin II receptor blockers (ARBS), beta-blockers, mineralocorticoid receptor antagonists, diuretics and digoxin), medications to treat arrhythmias (e.g., beta blockers and calcium channel blockers), medications to prevent coronary heart disease and cerebrovascular disease (e.g., statins and icosapent ethyl), and medications to treat and prevent thrombosis (e.g., antiplatelet agents, anticoagulants, warfarin, aspirin, dabigatran, apixaban, rivaroxaban and heparin).

F. Pancreas or Anti-Diabetic Agents

In some embodiments, the one or more additional therapeutic agents comprises a pancreas or anti-diabetic agent. The interchangeable terms “pancreas agent,” “pancreas drug,” “anti-diabetic agent,” or “anti-diabetic drug” (e.g., a second pancreas agent) herein refer to a drug or agent that is capable of treating, preventing, or reducing the risk of developing a pancreas disease or disorder, or a risk factor or symptom thereof, in a subject. In some embodiments, the second pancreas agent is an oral pancreas agent. In other embodiments, the second pancreas agent is a topical pancreas agent. In yet other embodiments, the second pancreas agent is an injectable pancreas agent. Pancreas agents herein can include, without limitation, type 2 diabetes medications (e.g., biguanides, sulfonylureas, meglitinides, thiazolidinediones, dipeptidyl peptidase-4 (DPP4) inhibitors, glucagon-like peptide-1 (GLP-1) receptor agonists, sodium-glucose co-transporter-2 (SGLT2) inhibitors and insulin), type 1 diabetes medications (e.g., insulin), medications to treat pancreatitis, and medications to treat pancreatic cancer (e.g., gemcitabine, 5-fluorouracil, oxaliplatin, paclitaxel, cisplatin, capecitabine and irinotecan).

G. Blood Agents

In some embodiments, the one or more additional therapeutic agents comprises a blood agent. The interchangeable terms “blood agent” or “blood drug” (e.g., a second blood agent) herein refer to a drug or agent that is capable of treating, preventing, or reducing the risk of developing a blood disease or disorder, or a risk factor or symptom thereof, in a subject. In some embodiments, the second blood agent is an oral blood agent. In other embodiments, the second blood agent is a topical blood agent. In yet other embodiments, the second blood agent is an injectable blood agent. Blood agents herein can include, without limitation, anemia treatments (e.g., iron supplements, vitamins, blood transfusion, erythropoietin, corticosteroids, immunoglobulins, and rituximab), medications to treat sickle cell disease (e.g., hydroxyurea, L-glutamine, voxelotor, crizanlizumab and non-steroidal anti-inflammatory drugs), medications to treat hemoglobinopathies (e.g., alpha-thalassemia and beta-thalassemia), medications to treat Hodgkin's lymphoma, medications to treat non-Hodgkin's lymphoma, medications to treat multiple myeloma and medications to treat leukemia.

H. Colon Agents

In some embodiments, the one or more additional therapeutic agents comprises a colon agent. The interchangeable terms “colon agent” or “colon drug” (e.g., a second colon agent) herein refer to a drug or agent that is capable of treating, preventing, or reducing the risk of developing a colon disease or disorder, or a risk factor or symptom thereof, in a subject in some embodiments, the second colon agent is an oral colon agent. In other embodiments, the second colon agent is a topical colon agent. In yet other embodiments, the second colon agent is an injectable colon agent. In still other embodiments, the second colon agent is a suppository. Colon agents herein can include, without limitation, corticosteroids, biologic therapies, antibiotics, anti-inflammatory drugs (e.g., sulfasalazine), immune suppressants (e.g., TNF inhibitors), antidiarrheal drugs, and laxatives.

I. Antiviral Agents

In some embodiments, the one or more additional therapeutic agents comprise an antiviral agent. Non-limiting examples of an antiviral agent include remdesivir (e.g., Veklury®), favipiravir (e.g., Avigan®), lopinavir/ritonavir (e.g., Kaletra®, Aluvia®), nitazoxanide (e.g., Alinia®), danoprevir (e.g., Ganovo®), ASC-09, umifenovir (e.g., Arbidol®), nafamostat, brequinar, AT-527, ABX464, merimepodib, molnupiravir, opaganib (e.g., Yeliva®), ivermectin (e.g., Soolantra®, Stromectol®, Sklice®), hydroxychloroquine, PF-07321332, PF-07321332/Ritonavir, casirivimab (e.g., REGN10933), imdevimab (e.g., REGN10987), casirivimab/imdevimab (e.g., REGEN-COV2), GT0918, AZD7442, etesevimab, and bamlanivimab, sotrovimab.

In some embodiments, the one or more additional therapeutic agents comprise an anti-inflammatory agent. Non-limiting examples of an anti-inflammatory agent include ruxolitinib (e.g., Jakafi®), baricitinib (e.g., Olumiant®), dapagliflozin (e.g., Farxiga®), EPA (in free acid or ethyl ester form, e.g., Lovaza®, Epadel®, Vascepa®), tocilizumab (e.g., Actemra®), sarilumab (e.g., Kevzara®), ravulizumab (e.g., Ultomiris®), losmapimod, pacritinib, bucillamine, tradipitant, lenzilumab, CD24Fc, acalabrutinib (e.g., Calquence®), otilimab, LY3127804, abivertinib maleate, BLD-2660, selinexor (e.g., Xpovio®), brequinar, PTC299, ibudilast, apilimod dimesylate, gimsilumab, OP-101, APN01, dociparastat sodium, itolizumab (Alzumab), pemziviptadil, prednisolone, dexamethasone, reparixin, brensocatib, emapalumab, and anakinra.

In some embodiments, the one or more additional therapeutic agents comprise an antimalaria agent. Non-limiting examples of an antimalaria agent include hydroxychloroquine and chloroquine.

In some embodiments, the one or more additional therapeutic agents comprise a biologic agent. In some embodiments, the biological agent is an antibody, for example, an antibody recognizing at least a portion of the SARS-CoV, MERS-CoV, and/or SARS-CoV-2 coronaviruses, such as an epitope on a spike protein.

In some embodiments, the biological agent is a vaccine, for example, a vaccine against the SARS-CoV, MERS-CoV, and/or SARS-CoV-2 coronaviruses. In some embodiments, the vaccine is BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), and/or NVX-CoV2373 (Novavax).

### Methods

Any composition of the present disclosure, including compositions described herein above or compositions that can be formulated from combining various embodiments of the present technology, can be used in treatment or prevention of a disease or disorder of the skin, kidney, liver, spleen, lung, heart, pancreas, blood, colon, and/or viral infection.

In some embodiments, the disease or disorder is associated with an organ or tissue selected from the group consisting of skin, kidney, liver, spleen, lung, heart, pancreas, blood, and/or colon.

In various embodiments, compositions of the present disclosure are useful for treatment and/or prevention of a skin disease. The term skin disease or disorder means any undesirable skin condition or symptom suffered by patients. Non-limiting examples of skin diseases/disorders include acne (e.g., vulgaris, rosacea), atopic dermatitis, bacterial infections, dermatitis, dry skin, eczema, fungal infections, photoprotection, psoriasis, pruritus/itch, photoprotection, radiation protection, seborrheic dermatitis, shingles, vasculitis, viral infections, and wrinkles.

In various embodiments, compositions of the present disclosure are useful for treatment and/or prevention of a renal disease/disorder. Non-limiting examples of renal diseases/disorders include polycystic kidney disease, chronic kidney disease, acute kidney failure, kidney infection (pyelonephritis), and kidney stones.

In various embodiments, compositions of the present disclosure are useful for treatment and/or prevention of a liver disease/disorder. Non-limiting examples of liver diseases/disorders include steatohepatitis (or non-alcoholic steatohepatitis, NASH), alcoholic hepatitis, liver toxicity, viral infection of the liver, viral hepatitis, autoimmune hepatitis, cryptogenic cirrhosis, hepatic necrosis following hypoperfusion, and hepatitis resulting from other disease, and secondary NASH.

In various embodiments, compositions of the present disclosure are useful for treatment and/or prevention of a spleen disease/disorder. Non-limiting examples of spleen diseases/disorders include splenomegaly, spleen cancer, asplenia, spleen trauma, idiopathic purpura, Felty's syndrome, Hodgkin's disease, and immune-mediated destruction of the spleen.

In various embodiments, compositions of the present disclosure are useful for treatment and/or prevention of a lung disease/disorder. Non-limiting examples of lung diseases/disorders include reactive airway disease, asthma, emphysema, COPD, respiratory tract infection, pleural cavity disease, pulmonary vascular disease, pneumonia, pulmonary embolism, lung cancer, and silicosis.

In various embodiments, compositions of the present disclosure are useful for treatment and/or prevention of a disease/disorder of the heart. Non-limiting examples of diseases/disorders of the heart include arrhythmia, atherosclerosis, cardiomyopathy, congenital heart defects, coronary artery disease (CAD), myocardial infarction, high blood pressure, cardia arrest, congestive heart failure, peripheral artery diseases, stroke, and heart infections.

In various embodiments, compositions of the present disclosure are useful for treatment and/or prevention of a disease/disorder of the pancreas. Non-limiting examples of diseases/disorders of the pancreas include type 1 diabetes, type 2 diabetes, pancreatitis, and pancreatic cancer.

In various embodiments, compositions of the present disclosure are useful for treatment and/or prevention of a disease/disorder of the blood. Non-limiting examples of diseases/disorders of the blood include anemia, hemoglobinopathies, sickle cell disease, alpha-thalassemia, beta-thalassemia, Hodgkin's lymphoma, non-Hodgkin's lymphoma, leukemia, and multiple myeloma.

In various embodiments, compositions of the present disclosure are useful for treatment and/or prevention of a disease/disorder of the colon. Non-limiting examples of diseases/disorders of the colon include inflammation, ulcerative colitis, colon cancer, colonic polyps, Crohn's disease, diverticulosis, diverticulitis, intestinal obstructions, and irritable bowel syndrome.

In various embodiments, compositions of the present disclosure are useful for treatment and/or prevention of infections and/or diseases caused by a viral infection. The viral infection can be caused by coronaviruses, including SARS caused by SARS-CoV and COVID-19 caused by SARS-CoV-2. As used herein, the terms “SARS-CoV-2”, “coronavirus”, “corona”, “2019 novel coronavirus,” “2019-nCoV”, and “COVID-19” are used interchangeably throughout the present disclosure. Non-limiting examples of coronaviruses include SARS-CoV, MERS-CoV, and SARS-COV-2. Non-limiting examples of diseases associated with a coronavirus include SARS, MERS, and COVID-19.

In another embodiment, the present disclosure provides methods for treatment, prevention, or amelioration of one or more symptoms and/or diseases associated with a coronavirus. In some embodiments, the methods further comprise monitoring the subject for SARS, MERS, COVID-19, and/or symptoms of SARS, MERS, or COVID-19. Non-limiting examples of symptoms of SARS-CoV infection and/or SARS include coughing, fever or chills, muscle pain, lethargy, sore throat, shortness of breath or difficulty in breathing, and pneumonia. Non-limiting examples of symptoms of MERS-CoV infection and/or MERS include coughing, fever or chills, expectoration, diarrhea, and shortness of breath or difficulty in breathing. Non-limiting examples of symptoms of SARS-CoV-2 infection and/or COVID-19 include coughing, fever or chills, muscle pain, fatigue, headache, shortness of breath or difficulty in breathing, loss of taste or smell, sore throat, congestion or runny nose, nausea or vomiting, and diarrhea.

In some embodiments, methods for treatment and/or prevention of a disease/disorder or symptoms associated thereof caused by SARS-CoV-2 in a subject are provided, wherein the subject is elderly (e.g., 65 years or greater), an infant, or an immunocompromised subject. In some embodiments, the subject has one or more underlying medical conditions resulting an increased risk of severe illness from COVID-19. Non-limiting examples of underlying medical conditions that render a subject at increased risk of severe illness from COVID-19 include cancer, cardiovascular disease, chronic kidney disease, chronic obstructive pulmonary disease (COPD), immunocompromised state, obesity (i.e., body mass index (BMI) of 30 or higher), serious heart conditions (e.g., heart failure, coronary artery disease, cardiomyopathy), sickle cell disease, Type 2 diabetes mellitus, asthma, cerebrovascular disease, cystic fibrosis, hypertension or high blood pressure, neurologic conditions (e.g., dementia), liver disease, pregnancy, pulmonary fibrosis, smoking, thalassemia, and Type 1 diabetes mellitus.

In some embodiments, methods of the present disclosure for treating or preventing a disease, for example, a disease of the skin, kidney, liver, spleen; lung, heart, pancreas, colon, and/or viral infection in a subject in need thereof comprise administering to the subject a therapeutically effective amount of an orally deliverable composition of 15-HETrE.

As used herein, the term “subject” refers to a mammalian subject, preferably a human. The phrases “subject” and “patient” are used interchangeably herein.

The term “treating” or “treatment” in relation to a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “preventing” or “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

In various embodiments, compositions of the present disclosure are administered in an amount sufficient to provide a daily 15-HETrE dose of about 1 mg to about 10,000 mg, about 25 mg to about 5000 mg, about 50 mg to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg, about 2600 mg, about 2700 mg, about 2800 mg, about 2900 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg, 5600 mg, about 5700 mg, about 5800 mg, about 5900 mg, about 6000 mg, about 6100 mg, about 6200 mg, about 6300 mg, about 6400 mg, about 6500 mg, 6600 mg, about 6700 mg, about 6800 mg, about 6900 mg, about 7000 mg, about 7100 mg, about 7200 mg, about 7300 mg, about 7400 mg, about 7500 mg, 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, about 9000 mg, about 10000 mg, about 10100 mg, about 10200 mg, about 10300 mg, about 10400 mg, about 10500 mg, about 10600 mg, about 10700 mg, about 10800 mg, about 10900 mg, about 11000 mg, about 11100 mg, about 11200 mg, about 11300 mg, about 11400 mg, about 11500 mg, about 11600 mg, about 11700 mg, about 11800 mg, about 11900 mg, or about 12000 mg of 15-HETrE per day.

In one embodiment, the method comprises administering to a subject in need of such treatment 15-HETrE in an amount of about 100 mg to about 8 g per day, about 300 mg to about 5 g per day, about 500 mg to about 3 g per day, about 1 g to about 2.5 g per day, about 1 g per day or about 2 g per day. In one embodiment, the 15-HETrE is administered to the subject daily for a period of at least about 2 weeks, at least about 4 weeks or at least about 8 weeks. In a related embodiment, upon treatment in accordance with the present disclosure, for example over a period of about 1 to about 12 weeks, about 1 to about 8 weeks, or about 1 to about 4 weeks, the subject or subject group exhibits a reduction or elimination of one or more symptoms of the disease.

In one embodiment, compositions of the present disclosure comprising 15-HETrE can be administered with food or without food. The term “with food” refers to, for example, ingesting the composition comprising 15-HETrE mixed with food, at the same time as food (e.g., a meal), immediately before or after ingesting food, within five minutes of ingesting food, within ten minutes of ingesting food, within fifteen minutes of ingesting food, or within thirty minutes of ingesting food.

In another embodiment, upon treatment with a composition of the present disclosure after a single dose administration or multiple dose administration, for example over a period of about 1 to about 12 weeks, about 1 to about 8 weeks, or about 1 to about 4 weeks, the subject or subject group exhibits a reduction or elimination of any 2 or more, any 3 or more, any 4 or more, any 5 or more, any 6 or more, any 7 or more, any 8 or more, any 9 or more, any 10 or more, any 11 or more symptoms of the disease.

In some embodiments, compositions of the present disclosure can be co-administered or administered concomitantly with one or more additional agents. The terms “co-administered,” “concomitant administration,” and “administered concomitantly” are used interchangeably herein and each refer to, for example, administration of two or more agents (e.g., 15-HETrE and a second, for example, skin agent) at the same time, in the same dosage unit, one immediately after the other, within five minutes of each other, within ten minutes of each other, within fifteen minutes of each other, within thirty minutes of each other, within one hour of each other, within two hours of each other, within four hours of each other, within six hours of each other, within twelve hours of each other, within one day of each other, within one week of each other, within two weeks of each other, within one month of each other, within two months of each other, within six months of each other, within one year of each other, etc.

In the various embodiments of the present disclosure described herein, the 15-HETrE and optional one or more additional agents can be administered as a co-formulated single dosage unit, or as individual dosage units. Where the 15-HETrE and optional one or more additional agents are co-administered as separate dosage units, each dosage unit can be administered to a subject at substantially the same or substantially different times. In one embodiment, where two or more individual dosage units are to be administered daily, each dosage unit can be administered to the subject within a period of about 24 hours, 18 hours, 12 hours, 10 hours, 8 hours, 6 hours, 4 hours, 2 hours, 1 hour, or 0.5 hours.

In another embodiment, the 15-HETrE and one or more additional therapeutic agents can be administered sequentially. For example, 15-HETrE can be administered to a subject as a sole agent during a 15-HETrE loading period. The loading period can be, for example, 1 day, 2 days, 4 days, 6 days, 2 weeks, 3 weeks, 4 weeks, 5 weeks, 6 weeks, 7 weeks, 8 weeks, 9 weeks, or 10 weeks. After any such loading period, one or more additional agents can be initiated together with current 15-HETrE administration or in place of 15-HETrE treatment.

In another embodiment, 15-HETrE is administered to a subject in the morning, for example from about 4 am to about 10 am, about 5 am to about 9 am, or about 6 am to about 8 am, and the optional one or more agents are administered to the subject in the afternoon or evening, for example from about 1 pm to about 11 pm, about 2 pm to about 10 pm, or about 3 pm to about 9 pm, or vice versa.

In another embodiment, the present disclosure provides the use of 15-HETrE and, optionally, one or more additional therapeutic agents in the manufacture of a medicament for treatment or prevention of a disease or disorder of the skin, kidney, liver, spleen, lung, pancreas, colon, and/or viral infection. In one embodiment, the composition contains not more than 10% DGLA, if any. In another embodiment, the composition contains substantially no or no DGLA.

In another embodiment, the present disclosure provides a pharmaceutical composition comprising 15-HETrE and, optionally, one or more additional therapeutic agents for the treatment and/or prevention of a disease or disorder of the skin, kidney, liver, spleen, lung, pancreas, colon, and/or viral infection wherein the composition contains not more than 10% DGLA, if any. In a related embodiment, the composition contains substantially no DGLA or no DGLA.

In some embodiments, the one or more additional therapeutic agents are administered to the subject in a range of from about 1 mg/kg to about 500 mg/kg, 10 mg/kg to about 150 mg/kg, from 30 mg/kg to about 120 mg/kg, from 60 mg/kg to about 90 mg/kg. In some embodiments, the one of more additional therapeutic agents are administered to the subject at a dose of about 15 mg/kg, about 30 mg/kg, about 45 mg/kg, about 60 mg/kg, about 75 mg/kg, about 90 mg/kg, about 105 mg/kg, about 120 mg/kg, about 135 mg/kg, or about 150 mg/kg. In some embodiments, the one of more additional therapeutic agents may be administered of once or multiple times a day.

It is within the purview of one of ordinary skill in the art to select a suitable administration route, such as oral administration, subcutaneous administration, intravenous administration, intramuscular administration, intradermal administration, intrathecal administration, or intraperitoneal administration, for the one or more additional therapeutic agents.

In some embodiments, the methods comprise administering to the subject a pharmaceutical composition comprising 15-HETrE and/or derivative thereof, and optionally one or more additional therapeutic agents, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the pharmaceutical composition comprising 15-HETrE and/or derivative thereof, and optionally one or more additional therapeutic agents, can be administered every day, every other day, every third day, weekly, biweekly (i.e., every other week), every third week, monthly, every other month, or every third month. The pharmaceutical composition comprising 15-HETrE and/or derivative thereof, and optionally one or more additional therapeutic agents, may be administered over a pre-determined time period. Alternatively, the pharmaceutical composition comprising 15-HETrE and/or derivative thereof, and optionally one or more additional therapeutic agents, may be administered until a particular therapeutic benchmark is reached. In some embodiments, the methods provided herein include a step of evaluating one or more therapeutic benchmarks in a biological sample, such as, but not limited to, the presence or absence of a virus or symptoms associated thereof, to determine whether to continue administration of the pharmaceutical composition comprising 15-HETrE and/or derivative thereof, and optionally one or more additional therapeutic agents.

In some embodiments, the subject is administered the one or more additional therapeutic agents before administration of the pharmaceutical composition comprising 15-HETrE and/or derivative thereof. In some embodiments, the subject is co-administered the one or more additional therapeutic agents and the pharmaceutical composition comprising 15-HETrE and/or derivative thereof. In some embodiments, the subject is administered the one or more additional therapeutic agents before after administration of the pharmaceutical composition comprising 15-HETrE and/or derivative thereof.

As one of ordinary skill in the art would understand, the pharmaceutical composition comprising 15-HETrE and/or derivative thereof and the one or more additional therapeutic agents can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

In one embodiment, the present disclosure provides a method of reducing or preventing side effects associated with administration of a second agent (e.g., skin agent). Administration of certain second agents disclosed herein have been associated with various intolerance symptoms, such as but not limited to: irritation of the oropharynx, mouth, mucus tissues, etc.; headache; nausea; upset stomach; severe allergic reactions (rash; hives; difficulty breathing; tightness in the chest; swelling of the mouth, face, lips, or tongue); blood in the urine; bloody vomit; diarrhea; dizziness; excitability; fast breathing; fast/irregular heartbeat; flushing; increased thirst or urination; irritability; muscle twitching; pounding in the chest; restlessness; seizures; stomach pain; trouble sleeping; and vomiting. In one embodiment, a method of reducing side effects associated with administration of a second agent as disclosed herein comprises discontinuing administration of a first pharmaceutical composition comprising the second agent and administering to a subject a second pharmaceutical composition comprising 15-HETrE as disclosed herein. In one embodiment, the second pharmaceutical composition includes an amount of a second agent that is less than the amount of the same second agent in the first pharmaceutical composition. In one embodiment, the second pharmaceutical composition includes an amount of the second agent that is about equal to or equal to the amount of that second agent in the first pharmaceutical composition. In one embodiment, the second pharmaceutical composition includes an amount of the second agent that is more than the amount of that second agent in the first pharmaceutical composition. In one embodiment, the second pharmaceutical composition does not include the second agent, essentially none of the second agent, or substantially none of the second agent.

In some embodiments, the methods of treating and/or preventing an infection caused by a viral infection (e.g., a coronavirus) comprise administering to a subject in need thereof a pharmaceutical composition comprising 15-HEPE and/or derivative thereof, and optionally one or more additional therapeutic agents (e.g., anti-viral agent), wherein upon administration the subject exhibits one or more of following outcomes listed in (a)-(h):

(a) a reduction in coughing, fever or chills, muscle pain, lethargy, fatigue, sore throat, expectoration, shortness of breath or difficulty in breathing, headache, loss of taste or smell, congestion or runny nose, nausea or vomiting, and/or diarrhea compared to baseline or control;

(b) a reduction in a risk for developing systemic inflammatory response syndrome (SIRS) compared to baseline or control;

(c) a reduction in a risk for developing acute respiratory distress syndrome (ARDS) compared to baseline or control;

(d) a reduction in a risk for developing sepsis compared to baseline or control;

(e) a reduction in respiratory conditions compared to baseline or control. Non-limiting examples of respiratory conditions include atelectasis, bronchiectasis, cough, emphysema, nasopharyngitis, orthopnea, pulmonary edema, wheezing, fibrotic lung disease, and pulmonary vascular disease;

(f) a reduction in viral RNA compared to baseline or control;

(g) a reduction in hospitalization compared to baseline or control; and/or

(h) a reduction in death compared to baseline or control.

In one embodiment, the methods comprise measuring baseline levels of one or more symptoms, risks, or conditions set forth in (a)-(h) above prior to dosing the subject or subject group. In another embodiment, the methods comprise administering a composition of the present disclosure to the subject after baseline levels of one or more symptoms, risks, or conditions set forth in (a)-(h) are determined, and subsequently taking an additional measurement of said one or more symptoms, risks, or conditions.

In another embodiment, upon treatment with a composition according to various embodiments disclosed herein, the subject exhibits one or more of:

(a) a reduction in coughing, fever or chills, muscle pain, lethargy, fatigue, sore throat, expectoration, shortness of breath or difficulty in breathing, headache, loss of taste or smell, congestion or runny nose, nausea or vomiting, and/or diarrhea of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control;

(b) a reduction in a risk for developing SIRS of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control;

(c) a reduction in a risk for developing ARDS of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control;

(d) a reduction in a risk for developing sepsis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control; and

(e) a reduction in respiratory conditions, including atelectasis, bronchiectasis, cough, emphysema, nasopharyngitis, orthopnea, pulmonary edema, wheezing, fibrotic lung disease, and pulmonary vascular disease, of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control;

(f) a reduction in viral RNA of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to baseline or control;

(g) a reduction in hospitalization of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to baseline or control; and/or

(h) a reduction in death of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to baseline or control.

Itis to be understood that a wide range of changes and modifications to the embodiments described above will be apparent to those skilled in the art and are contemplated. It is, therefore, intended that the foregoing detailed description be regarded as illustrative rather than limiting, and that it be understood that it is the following claims, including all equivalents, that are intended to define the spirit and scope of the present disclosure.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims. Further, several lists of components and their amounts have been mentioned above. The present disclosure of the present disclosure includes a selection from any one of these lists combined with any other selection(s) from any of the lists. It is not practicable to write out each combination. It will readily be appreciated that a compositions of the present disclosure have a number of components and for each, the description provides guidance as to the relative amounts and purity for example of that component in the overall composition.

### EXAMPLES

### Example 1: Tissue Distribution Following Oral Administration of 15-HETrE

The objective of this study was to examine distribution of orally administered 15-HETrE. Male CRL:CD rats were obtained from Charles River Laboratories. Upon receipt, animals were allowed to acclimatize for 5 days prior to start of testing, housed under a 12 h light/dark cycle, and provided a pellet diet (RM-1) and water ad libitum. Animals were randomized into 2 groups of 5 mice each and received a daily dosing each day as provided in Table 1. Animals were dosed with 15-HETrE or vehicle (0.5% HPMC in ASTM Type 1 water) p.o. once daily for 7 days. Animals were monitored at 0.25, 0.5, and 1 hour post dose. During the treatment period, individual body weight was measured daily as well as clinical signs of behavior and survival.

TABLE 1

| Group No. | No. of animals | Treatment | Dose |
|---|---|---|---|
| 1 | 5 | Vehicle | 5 ml/kg |
| 2 | 5 | 15-HETrE | 500 mg/kg/5 ml |

24 hours after the final p.o. dose, each animal was killed by anaesthetic overdose with an intraperitoneal injection of pentobarbitone. A blood sample was collected from the descending vena cava. Lungs, heart, liver, kidneys, spleen, colon, and an area of shaved skin were harvested from each animal, rinsed in PBS, dabbed dry, weighed, and snap-frozen by immersion in liquid nitrogen. All tissue samples were stored frozen at −80° C.

### Results

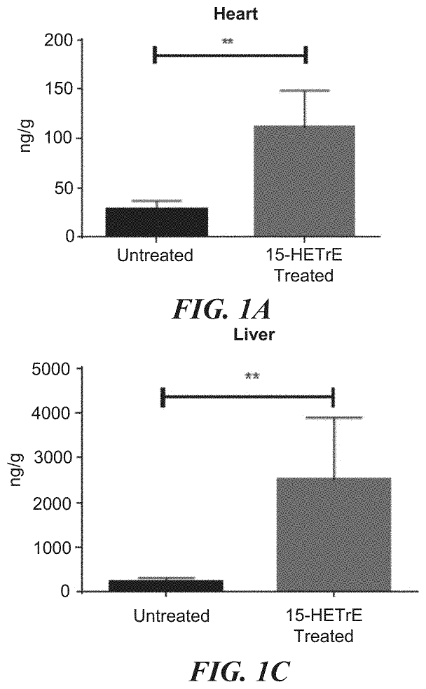

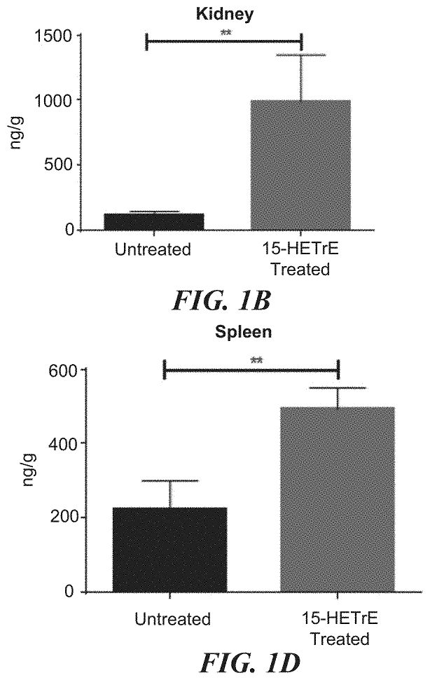

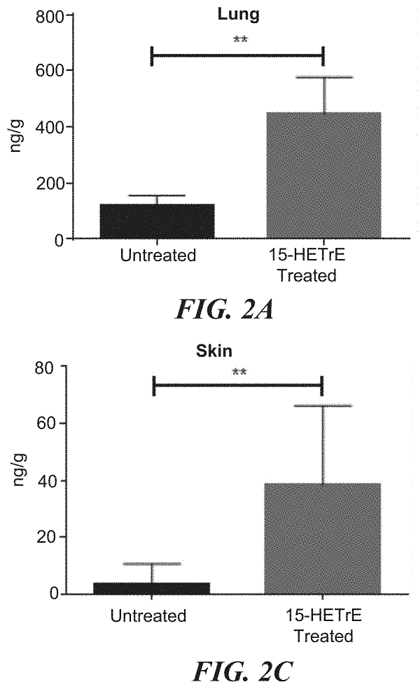

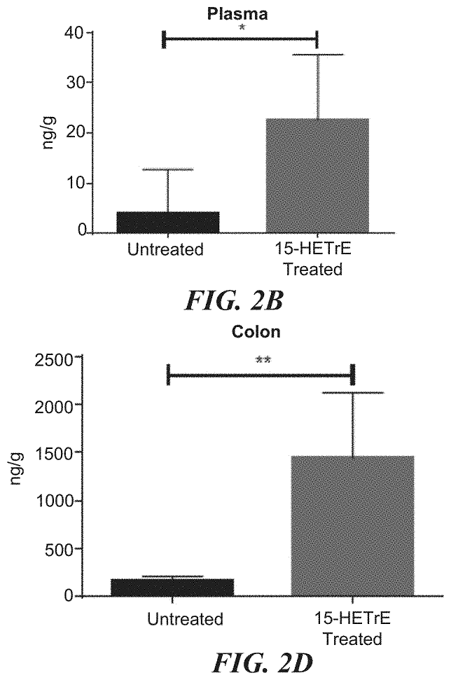

The summary of the analysis is given in FIGS. 1A-1D and FIGS. 2A-2D. FIGS. 1A-1D show comparison of the level of 15-HETrE in the heart, kidney, liver, and spleen of 15-HETrE-treated animals as compared to control animals. FIGS. 2A-2D shows comparison of the level of 15-HETrE in the lung, plasma, skin, and colon of 15-HETrE-treated animals as compared to control animals. Interestingly, orally administered 15-HETrE targeted all organs tested, including the skin.

### Example 2: Alkyl (Ethyl) Ester of 15-HETrE Synthetic Route from 15-HETrE Free Fatty Acid

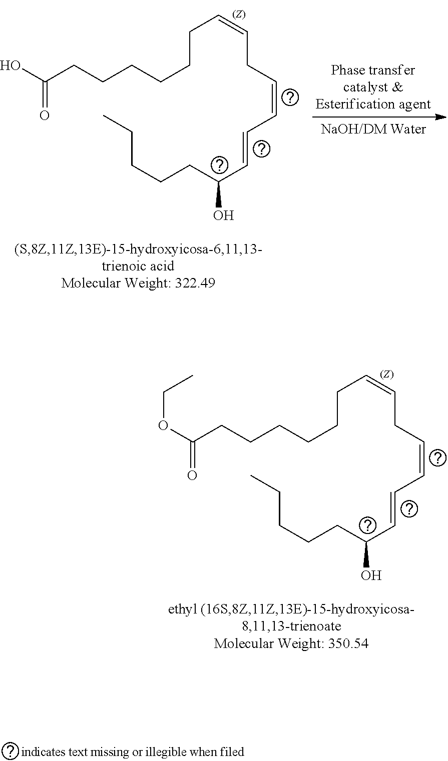 [SMILES: CCCCC[C@H](O)/C=C/C=C\C/C=C\CCCCCCC(=O)O ; CCCCC[C@H](O)/C=C/C=C\C/C=C\CCCCCCC(=O)OCC]

This example shows a method of synthesizing 15-HETrE ethyl ester from 15-HETrE free acid. Sodium hydroxide (19.1 g) and demineralized water (700 mL) were charged into a clean 3 L multi-neck RB flask. The solution was stirred well and then cooled to 20 to 30° C. To the caustic solution was added a solution of 15(S)-HETrE (140 g) dissolved in MTBE (2808 g) and the mass was concentrated under vacuum to distill off MTBE. To the stirring solution were added a suitable phase transfer catalyst, followed by the addition of a compatible esterification agent. The reaction mixture was then stirred as required at suitable temperature. After achieving the desired conversion, the reaction mixture was extracted with n-hexane (1400 mL). The organic layer was washed with purified water (700 mL) and 15% brine solution (420 mL). The separated organic layer was dried over anhydrous sodium sulfate (28 g) and filtered. The filtrate was taken for charcoal treatment with norit SX plus carbon (14 g) at suitable temperature and filtered. The filtrate was treated with silica gel (100-200 mesh, 28 g) at suitable temperature and filtered and 0.35% dL-α-tocopherol was added (based on weight of 15-HETRE EE in filtrate). The filtrate was concentrated under vacuum to distill off solvent completely and chased with MTBE at suitable temperature to get 15(S)-HETrE EE product as pale-yellow liquid.

### Example 3: Alkyl (Ethyl) Ester of 15-HETrE—Tests and Results

In this example, the analysis results of 15-HETrE ethyl ester are shown below in Table 2:

TABLE 2
Analysis results

| | Batch number | | | | |
|---|---|---|---|---|---|
| | 2811/A0139/ | 2811/A0139/ | 2811/A0139/ | 2811/A0178/ | 2811/A0178/ |
| | A/II/018 | A/II/019 | A/II/020 | B/I/004 | B/I/010 |
| Test | | | | | |
| Appearance | Pale yellow | Pale yellow | Pale yellow | Pale yellow | Pale yellow |
| | viscous oil | viscous oil | viscous oil | viscous oil | viscous oil |
| Identification | | | | | |
| i) By IR | Conforms to | Conforms to | Conforms to | Conforms to | Conforms to |
| ii) By ¹H NMR | reference | reference | reference | reference | reference |
| | structure | structure | structure | structure | structure |
| Water content | 0.3 | 0.6 | 0.3 | 0.7 | 0.7 |
| by KF(% w/w) | | | | | |
| Assay by | 95.2 | 94.3 | 96.5 | 95.5 | 96.3 |
| HPLC(% w/w) | | | | | |
| Purity by | 93.8 | 93.2 | 94.8 | 93.0 | 94.1 |
| HPLC(% a/a) | | | | | |
| Chiral purity | 99.6 | 99.5 | 99.5 | 99.8 | 99.4 |
| by HPLC (% a/a) | | | | | |
| Residue on | 0.1 | 0.1 | 0.1 | 0.1 | 0.0 |
| ignition(% w/w) | | | | | |
| Residual solvents | | | | | |
| by GC-HS(ppm) | | | | | |
| i) Methyl tert | 261 | 4591 | 7 | 29 | ND |
| butyl ether | | | | | |
| ii) n-Hexane | 6 | 10 | 6 | 3 | 1 |
| Elemental | | | | | |
| impurities (ppm) | | | | | |
| a) Cadmium | ND | ND | ND | ND | ND |
| b) Lead | 0.04 | 0.03 | 0.02 | 0.1 | 0.06 |
| c) Arsenic | ND | ND | ND | ND | ND |
| d) Mercury | 0.09 | ND | ND | 0.05 | 0.01 |
| e) Cobalt | ND | ND | ND | ND | ND |
| f) Vanadium | ND | ND | ND | ND | ND |
| g) Nickel | ND | ND | ND | 0.02 | 3.59 |
| h) Molybdenum | ND | 0.02 | ND | ND | ND |
| i) Chromium | 0.02 | 0.08 | 0.05 | 0.03 | 0.02 |
| j) Manganese | 0.02 | 0.03 | 0.03 | 0.15 | 0.18 |

### Example 4: In Vitro Antiviral Activities of 15-HETrE Against SARS-CoV-2 Using the Viral Yield Reduction Assay

The objective of this example is to examine the antiviral activities of 15-HETrE against SARS-CoV-2 in vitro, either alone or in combination with co-treatment drugs including remdesivir and favipiravir.

A viral yield reduction assay will be conducted using Caco2 and VeroE6 cells. To test the individual antiviral activities, Caco2 cells will be infected with SARS-CoV-2 in the presence of different concentrations of the following treatments: (1) linoleic acid (5, 20, 50, and 100 μM); (2) 15-HETrE (5, 20, 50, and 100 μM); (3) remdesivir (6.4, 20, 64, and 200 nM); and (4) favipiravir (62, 124, 250, and 500 μM). Cell culture supernatants will be collected 72 hours post-infection for determination of endpoint titers in VeroE6 cells. 50% cytotoxic concentration (CC50) and 50% effective concentration (EC50) will be calculated.

Then, to test the combined antiviral activities, Caco2 cells will be infected with SARS-CoV-2 in the presence of different concentrations of the following combination treatments: (1) combination of remdesivir and linoleic acid; (2) combination of remdesivir and 15-HETrE; (3) combination of favipiravir and linoleic acid; and (4) combination of favipiravir and 15-HETrE. The test concentrations will depend on the outcome of the individual antiviral activities. Similarly, cell culture supernatants will be collected 72 hours post-infection for determination of endpoint titers in VeroE6 cells, and CC50 and EC50 will be calculated.

### Example 5: In Vitro Antiviral Activities of 15-HETrE Against SARS-CoV-2 Using the IC50 Assay with Optional qPCR

The objective of this example is to further characterize the antiviral activities of 15-HETrE against SARS-CoV-2 in vitro, either alone or in combination with co-treatment drugs including remdesivir and favipiravir.

To determine of the IC50 (i.e., half maximal inhibitory concentration) of the tested compounds, VeroE6 cells will be infected with SARS-CoV-2 in the presence of different concentrations of the following treatments: (1) linoleic acid (10 concentrations, 2-fold dilution starting from 100 μM); (2) 15-HETrE (10 concentrations, 2-fold dilution starting from 100 μM); (3) remdesivir (0.1-50 μM); and (4) favipiravir (10 concentrations, 2-fold dilution starting from 500 μM). For each treatment, duplicate samples will be prepared. After 24 hours of incubation, cells from one set of samples will be stained for positivity of virus, quantified, and IC50 will be calculated. Based on the IC50 results, certain duplicate samples will be selected for qPCR quantification of virus in supernatants. The duplicate samples will be incubated for total of 48 hours if the amount of virus is too low for quantification after 24 hours.

Using similar protocols, the IC50 will also be determined for the following combination treatments: (1) combination of remdesivir and linoleic acid; (2) combination of remdesivir and 15-HETrE; (3) combination of favipiravir and linoleic acid; and (4) combination of favipiravir and 15-HETrE. The test concentrations will depend on the outcome of the IC50 results from the individual testing.

### Example 6: In Vitro Activity of 15-HETrE on Diabetes

The aim of the following study was to determine the effects of 15(S)-HETrE activity on diabetes. This aim was accomplished by (1) identifying a cytotoxic threshold of 15(S)-HETrE in islet microtissues and (2) testing the effects of 15(S)-HETrE on human islet microtissue function under conditions of glucotoxicity and cytokine stress.

### A. Experimental Summary—Cytotoxicity Pretest

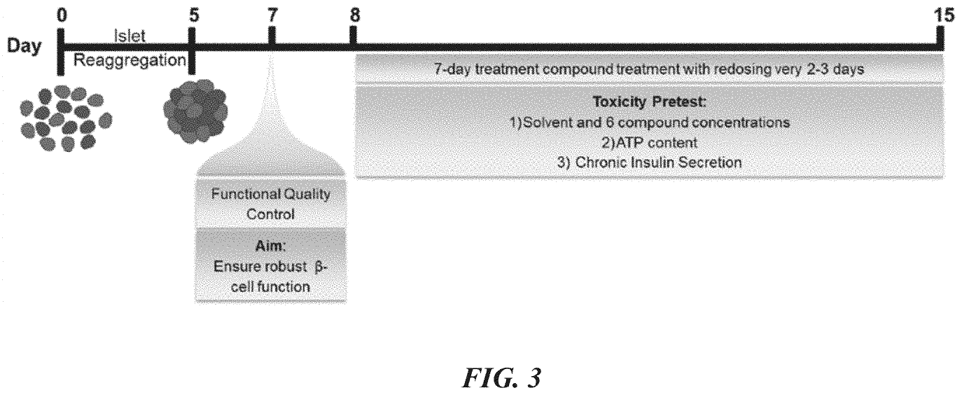

The schedule for this phase of the study is provided in FIG. 3. From Days 0-7, a human islet microtissue was established. On Day 7, 15-HETrE treatment commenced. From Days 9-12, the culture media was renewed and the compound (15-HETrE) was re-dosed. On Day 14, an assessment on islet cell viability was performed—tissues were lysed to measure ATP content using Promega CellTiter-Glo luminescent cell viability assay. On Day 14, an assessment on islet cell function was also performed—conditioned media from the last 48 hours of the experiment was collected to test chronic insulin secretion. And lastly, on Day 14, insulin was quantified—following dilutions in Krebs-Ringer HEPES buffer, chronic insulin secretion was quantified using STELLUX Chemi Human Insulin ELISA assay.

### B. Experimental Results—Cytotoxicity Pretest

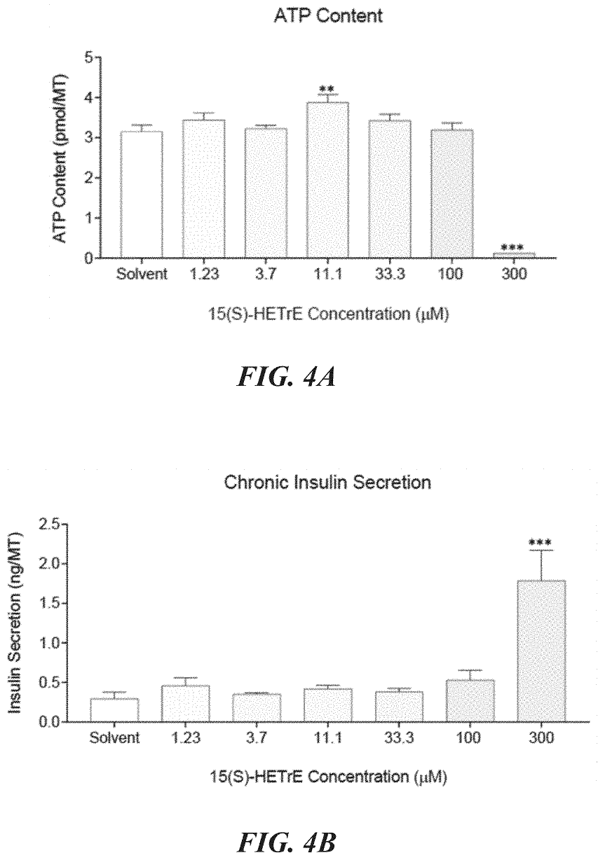

After 7 days of treatment with increasing concentrations of 15(S)-HETrE, the following results were observed: (1) there was a significant decrease in ATP content (FIG. 4A) after treatment with 300 μM 15(S)-HETrE; (2) there was a significant increase in ATP content when treated with 11.1 μM 15(S)-HETrE (FIG. 4b); and (3) there was a significant decrease chronic insulin secretion (FIG. 3B) after treatment with 300 μM 15(S)-HETrE. Based on these results, the concentrations 12.5 μM, 25 μM and 50 μM were selected for testing compound efficacy.

### C. Experimental Summary—15-HETrE Efficacy

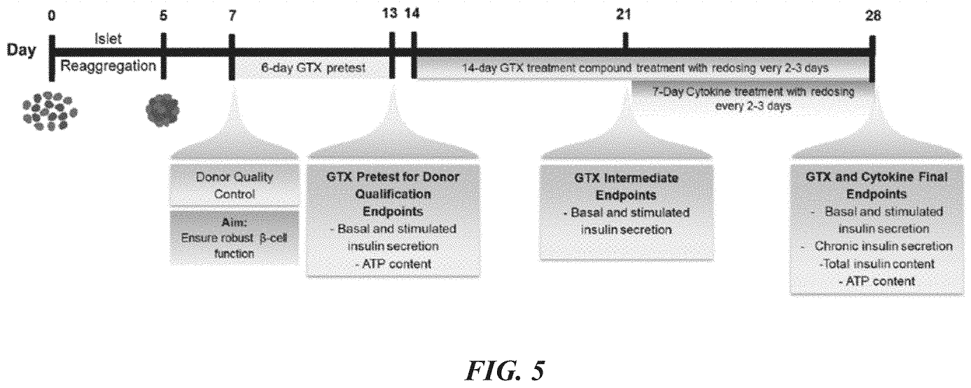

The schedule for this phase of the study is provided in FIG. 5. From Days 0-13, an islet microtissue model was established and glucotoxicity pre-test performed. Day 14 marked the start of glucotoxicity assay and 12.5 μM, 25 μM and 50 μM 15(S)-HETrE treatment. From Days 16-19, the culture media was renewed, and the compound (15-HETrE) was re-dosed. Day 12 marked the start of the cytokine stress assay for the 12.5 μM, 25 μM and 50 μM 15(S)-HETrE treatment. On Day, 12 an intermediate assessment of the compound effects was also performed (e.g., Day 7 incubation in glucotoxic conditions). On Day 12, a renewal of the culture media and compound treatment was performed. On Days 23 and 26, a renewal of the culture media and compound treatment was also performed. Lastly, on Day 28, an assessment of the compound effects under glucotoxicity/cytokine stress at the end of 14- and 7-day incubation was performed.

### D. Experimental Results—15-HETrE Efficacy

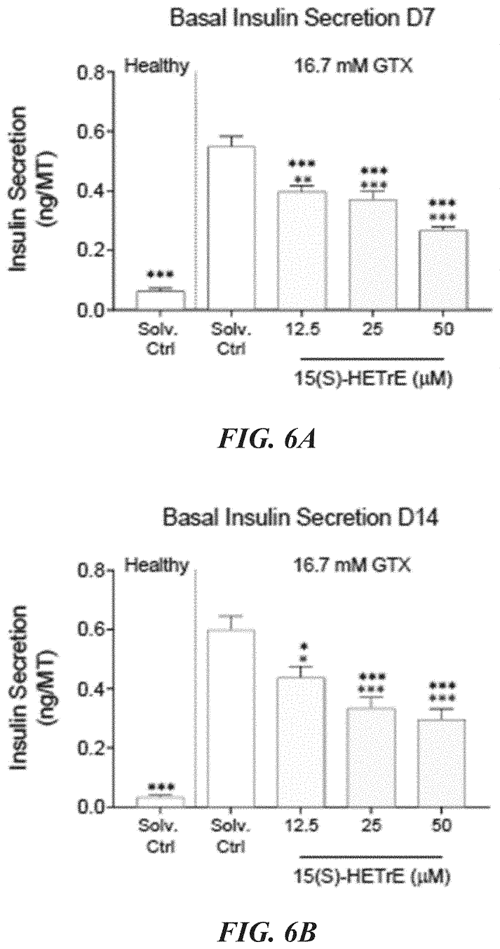

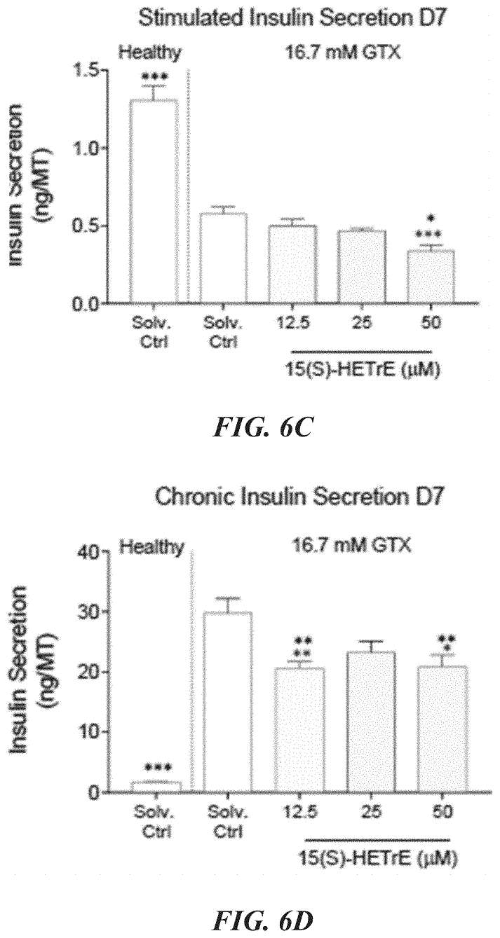

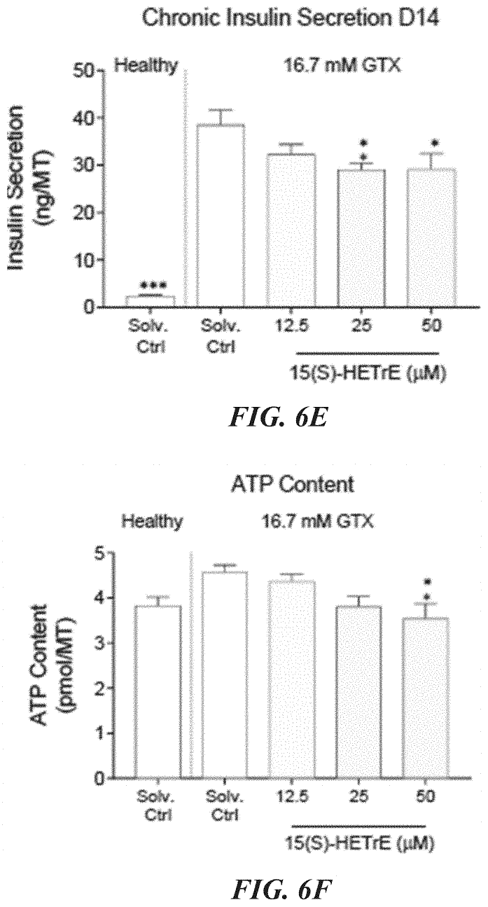

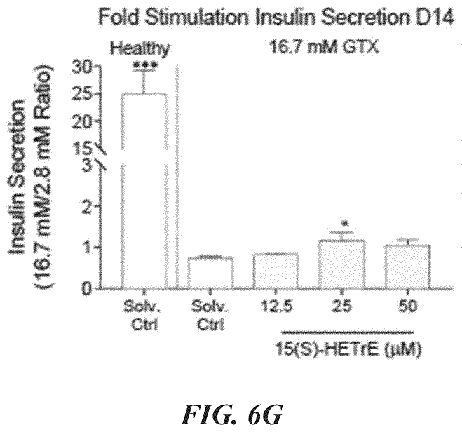

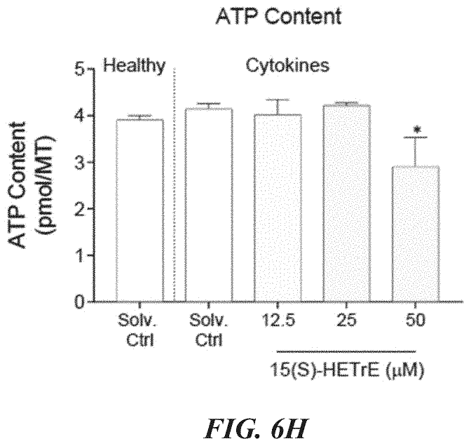

The results from this study showed that islet microtissues treated with 15(S)-HETrE under glucotoxic conditions exhibited: (1) dose-dependent decrease in basal insulin secretion at days 7 (FIG. 6A) and 14 (FIG. 6B); (2) a significant decrease in stimulated insulin secretion at day 7 with 50 μM 15(S)-HETrE (FIG. 6C); (3) reduced chronic insulin secretion at days 7 (FIG. 6D) and 14 (FIG. 6E); (4) significantly decreased ATP levels with 50 μM 15(S)-HETrE (FIG. 6F); and (5) improved fold stimulation of insulin secretion with 25 μM 15(S)-HETrE at day 14 (FIG. 6G).

The results from this study also showed that islet microtissues treated with 15(S)-HETrE under cytokine stress exhibited a significant decrease in ATP content with 50 μM 15(S)-HETrE (FIG. 5H).

Para A: A composition comprising 15-HETrE and or more active agents selected from the group consisting of a skin agent, a renal agent, a liver agent, a lung agent, a heart agent, a pancreas agent or anti-diabetic agent, a blood agent, a colon agent, and an anti-viral agent.

Para B: An orally deliverable composition comprising 15-HETrE.

Para C: The composition of Para A or Para B, wherein the 15-HETrE is in free acid form and/or a pharmaceutically acceptable ester, derivative, conjugate, or salt thereof, or mixtures of any of the foregoing.

Para D: The composition of Para C, wherein the 15-HETrE is 15-HETrE ethyl ester.

Para E: The composition as in any one of Paras A-D, wherein the 15-HETrE is present in an amount up to about 1500 mg.

Para F: The composition as in any one of Paras A-E, wherein the composition comprises up to about 4 g of 15-HETrE.

Para G: The composition as in any one of Paras A-E, wherein the composition comprises up to about 2 g of 15-HETrE.

Para H: The composition as in any one of Paras A-E, wherein the composition comprises about 2 g to about 4 g of 15-HETrE.

Para I: The composition as in any one of Paras A-E, wherein the 15-HETrE represents at least about 80%, by weight, of all fatty acids present in the composition.

Para J: The composition as in any one of Paras A-I, wherein the composition is present in a solid dosage form.

Para K: The composition of Para J, wherein the solid dosage form comprises a capsule.

Para L: The composition as in any one of Paras A-K, wherein the composition is present in a liquid dosage form or semi-solid dosage form.

Para M: The composition of Para L, wherein the liquid dosage form or semi-solid dosage form comprises a solution or an emulsion.

Para N: The composition of Para B, further comprising one or more therapeutic agents selected from the group consisting of a skin agent, a renal agent, a liver agent, a lung agent, a heart agent, a pancreas agent or anti-diabetic agent, a blood agent, a colon agent, and an anti-viral agent.

Para O: The composition of Para A, formulated for intravenous administration, oral administration, or intranasal administration.

Para P: A method of treating or preventing a disease in a subject in need thereof, the method comprising administering to the subject a composition comprising 15-HETrE, wherein the disease is selected from the group consisting of a skin disease, kidney disease, liver disease, spleen disease, lung disease, heart disease, pancreas disease, blood disease, colon disease, and a viral disease.

Para Q: The method of Para P, wherein the 15-HETrE is in free acid form and/or a pharmaceutically acceptable ester, derivative, conjugate, or salt thereof, or mixtures of any of the foregoing.

Para R: The method of Para Q, wherein the 15-HETrE is 15-HETrE ethyl ester.

Para S: The method as in any one of Paras P-R, wherein the composition comprises 15-HETrE in an amount up to about 1500 mg.

Para T: The method as in any one of Paras P-R, wherein the composition comprises up to about 4 g of 15-HETrE.

Para U: The method as in any one of Paras P-R, wherein the composition comprises up to about 2 g of 15-HETrE.

Para V: The method as in any one of Paras P-R, wherein the composition comprises about 2 g to about 4 g of 15-HETrE.

Para W: The method as in any one of Paras P-V, wherein the 15-HETrE represents at least about 80%, by weight, of all fatty acids present in the composition.

Para X: The method as in any one of Paras P-V, wherein the subject is administered one or more therapeutic agents selected from the group consisting of a skin agent, a renal agent, a liver agent, a lung agent, a heart agent, a pancreas agent or anti-diabetic agent, a blood agent, a colon agent, and anti-viral agent.

Para Y: The method as in any one of Paras P-X, wherein the composition is administered intravenously, orally, or intranasally.

Para Z: The method as in any one of Paras P-Y, wherein the skin disease is selected from the group consisting of acne, atopic dermatitis, bacterial infections, dermatitis, dry skin, eczema, fungal infections, photoprotection, psoriasis, pruritus/itch, photoprotection, radiation protection, seborrheic dermatitis, shingles, vasculitis, viral infections, and wrinkles.

Para AA: The method as in any one of Paras P-Y, wherein the renal disease is selected from the group consisting of polycystic kidney disease, chronic kidney disease, acute kidney failure, kidney infection (pyelonephritis), and kidney stones.

Para AB: The method as in any one of Paras P-Y, wherein the liver disease is selected from the group consisting of steatohepatitis, alcoholic hepatitis, liver toxicity, viral infection of the liver, viral hepatitis, autoimmune hepatitis, cryptogenic cirrhosis, hepatic necrosis following hypoperfusion, and hepatitis resulting from other disease, and secondary NASH.

Para AC: The method as in any one of Paras P-Y, wherein the spleen disease is selected from the group consisting of splenomegaly, spleen cancer, asplenia, spleen trauma, idiopathic purpura, Felty's syndrome, Hodgkin's disease, and immune-mediated destruction of the spleen.

Para AD: The method as in any one of Paras P-Y, wherein the lung disease is selected from the group consisting of reactive airway disease, asthma, emphysema, COPD, respiratory tract infection, pleural cavity disease, pulmonary vascular disease, pneumonia, pulmonary embolism, lung cancer, and silicosis.

Para AE: The method as in any one of Paras P-Y, wherein the heart disease is selected from the group consisting of arrhythmia, atherosclerosis, cardiomyopathy, congenital heart defects, coronary artery disease (CAD), myocardial infarction, high blood pressure, cardia arrest, congestive heart failure, peripheral artery diseases, stroke, and heart infections.

Para AF: The method as in any one of Paras P-Y, wherein the pancreas disease is selected from the group consisting of type 1 diabetes, type 2 diabetes, pancreatitis, and pancreatic cancer.

Para AG: The method as in any one of Paras P-Y, wherein the blood disease is selected from the group consisting of anemia, hemoglobinopathy, sickle cell disease, alpha-thalassemia, beta-thalassemia, Hodgkin's lymphoma, non-Hodgkin's lymphoma, leukemia, and multiple myeloma.

Para AH: The method as in any one of Paras P-Y, wherein the colon disease is selected from the group consisting of inflammation, ulcerative colitis, colon cancer, colonic polyps, Crohn's disease, diverticulosis, diverticulitis, intestinal obstructions, and irritable bowel syndrome.

Para AI: The method as in any one of Paras P-Y, wherein the viral disease is caused by a coronavirus.

Para AJ: The method of Para AI, wherein the coronavirus is selected from the group consisting of SARS-COV, MERS-COV, and SARS-COV-2.

From the foregoing, it will be appreciated that specific embodiments of the invention have been described herein for purposes of illustration, but that various modifications may be made without deviating from the scope of the invention. Accordingly, the invention is not limited except as by the appended claims.

1. A composition comprising 15-HETrE and or more active agents selected from the group consisting of a skin agent, a renal agent, a liver agent, a lung agent, a heart agent, a pancreas agent or anti-diabetic agent, a blood agent, a colon agent, and an anti-viral agent.

2. The composition of claim 1, wherein the 15-HETrE is in free acid form and/or a pharmaceutically acceptable ester, derivative, conjugate, or salt thereof, or mixtures of any of the foregoing.

3. The composition of claim 2, wherein the 15-HETrE is 15-HETrE ethyl ester.

4. The composition of claim 1, wherein the 15-HETrE is present in an amount up to about 1500 mg.

5. The composition of claim 1, wherein the composition comprises up to about 4 g of 15-HETrE.

6. The composition of claim 1, wherein the composition comprises up to about 2 g of 15-HETrE.

7. The composition of claim 1, wherein the composition comprises about 2 g to about 4 g of 15-HETrE.

8. The composition of claim 1, wherein the 15-HETrE represents at least about 80%, by weight, of all fatty acids present in the composition.

9. The composition of claim 1, wherein the composition is present in a solid dosage form.

10. The composition of claim 9, wherein solid dosage form comprises a capsule.

11. The composition of claim 1, wherein the composition is present in a liquid dosage form or semi-solid dosage form.

12. The composition of claim 11, wherein liquid dosage form or semi-solid dosage form comprises a solution or an emulsion.

13. The composition of claim 1, formulated for intravenous administration, oral administration, or intranasal administration.

14. A method of treating or preventing a disease in a subject in need thereof, the method comprising administering to the subject a composition comprising 15-HETrE, wherein the disease is selected from the group consisting of a skin disease, kidney disease, liver disease, spleen disease, lung disease, heart disease, pancreas disease, blood disease, colon disease, and a viral disease.

15. The method of claim 14, wherein the 15-HETrE is in free acid form and/or a pharmaceutically acceptable ester, derivative, conjugate, or salt thereof, or mixtures of any of the foregoing.

16. The method of claim 15, wherein the 15-HETrE is 15-HETrE ethyl ester.

17. The method of claim 14, wherein the composition comprises 15-HETrE in an amount up to about 1500 mg.

18. The method of claim 14, wherein the composition comprises up to about 4 g of 15-HETrE.

19. The method of claim 14, wherein the composition comprises up to about 2 g of 15-HETrE.

20. The method of claim 14, wherein the composition comprises about 2 g to about 4 g of 15-HETrE.

21. The method of claim 14, wherein the 15-HETrE represents at least about 80%, by weight, of all fatty acids present in the composition.

22. The method of claim 14, wherein the subject is administered one or more therapeutic agents selected from the group consisting of a skin agent, a renal agent, a liver agent, a lung agent, a heart agent, a pancreas agent or anti-diabetic agent, a blood agent, a colon agent, and anti-viral agent.

23. The method of claim 14, wherein the composition is administered intravenously, orally, or intranasally.

24. The method of claim 14, wherein the skin disease is selected from the group consisting of acne, atopic dermatitis, bacterial infections, dermatitis, dry skin, eczema, fungal infections, photoprotection, psoriasis, pruritus/itch, photoprotection, radiation protection, seborrheic dermatitis, shingles, vasculitis, viral infections, and wrinkles.

25. The method of claim 14, wherein the renal disease is selected from the group consisting of polycystic kidney disease, chronic kidney disease, acute kidney failure, kidney infection (pyelonephritis), and kidney stones.

26. The method of claim 14, wherein the liver disease is selected from the group consisting of steatohepatitis, alcoholic hepatitis, liver toxicity, viral infection of the liver, viral hepatitis, autoimmune hepatitis, cryptogenic cirrhosis, hepatic necrosis following hypoperfusion, and hepatitis resulting from other disease, and secondary NASH

27. The method of claim 14, wherein the spleen disease is selected from the group consisting of splenomegaly, spleen cancer, asplenia, spleen trauma, idiopathic purpura, Felty's syndrome, Hodgkin's disease, and immune-mediated destruction of the spleen.

28. The method of claim 14, wherein the lung disease is selected from the group consisting of reactive airway disease, asthma, emphysema, COPD, respiratory tract infection, pleural cavity disease, pulmonary vascular disease, pneumonia, pulmonary embolism, lung cancer, and silicosis.

29. The method of claim 14, wherein the heart disease is selected from the group consisting of arrhythmia, atherosclerosis, cardiomyopathy, congenital heart defects, coronary artery disease (CAD), myocardial infarction, high blood pressure, cardia arrest, congestive heart failure, peripheral artery diseases, stroke, and heart infections.

30. The method of claim 14, wherein the pancreas disease is selected from the group consisting of type 1 diabetes, type 2 diabetes, pancreatitis, and pancreatic cancer.

31. The method of claim 14, wherein the blood disease is selected from the group consisting of anemia, hemoglobinopathy, sickle cell disease, alpha-thalassemia, beta-thalassemia, Hodgkin's lymphoma, non-Hodgkin's lymphoma, leukemia, and multiple myeloma.

32. The method of claim 14, wherein the colon disease is selected from the group consisting of inflammation, ulcerative colitis, colon cancer, colonic polyps, Crohn's disease, diverticulosis, diverticulitis, intestinal obstructions, and irritable bowel syndrome.

33. The method of claim 14, wherein the viral disease is caused by a coronavirus.

34. The method of claim 33, the coronavirus is selected from the group consisting of SARS-CoV, MERS-CoV, and SARS-CoV-2.

35. An orally deliverable composition comprising 15-HETrE.
